(19) United States
(12) Patent Application Publication
(10) Pub. No.: US 2006/0177490 A1
(43) Pub. Date: Aug. 10, 2006

(54) Chitosan-base antimicrobial thermoplastic polymer blends

(76) Inventor: Debora Flanagan Massouda, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/349,486

(22) Filed: Feb. 7, 2006

Related U.S. Application Data
(60) Provisional application No. 60/650,652, filed on Feb. 7, 2005.

(51) Int. Cl. C08L 5/00; B32B 5/02; A61K 9/70; A61K 8/73

(52) U.S. Cl. 424/443; 524/27; 428/615; 442/152

(57) ABSTRACT

A method is provided to produce antimicrobial thermoplastic polymer blends by blending chitosan acid salts with polymers having amino-reactive functional groups. The antimicrobial thermoplastic polymer blends produced and articles composed of these materials are described.

This application claims priority to U.S. provisional application Ser. No. 60/650,652, filed Feb. 7, 2005, the entire disclosure of which is incorporated herein by reference.

This invention relates to the field of antimicrobial materials. Specifically, chitiosan-containing antimicrobial polymer blends and a method of producing these blends are provided.

### BACKGROUND OF THE INVENTION

A demand exists for materials and/or processes that either minimize or kill microorganisms encountered in the environment. Such materials are useful in connection with food packaging, preparation, and handling. They are also useful in applications related to personal hygiene, such as for garments and personal care articles, and in locations with high potential for microbial contamination such as bathrooms. Similarly, antimicrobial materials are useful in hospitals and nursing homes where people with lowered resistance are especially vulnerable to microorganisms. Antimicrobial thermoplastic polymer blends would be particularly useful in the manufacture of antimicrobial articles.

Chitosan compounds are known to provide antimicrobial activity as bacteriocides and fungicides (see, e.g., T. L. Vigo, “Antimicrobial Polymers and Fibers: Retrospective and Prospective,” in Bioactive Fibers and Polymers, J. V. Edwards and T. L. Vigo, eds., ACS Symposium Series 792, pp. 175-200, American Chemical Society, 2001). Chitosan is also known to impart antiviral activity, though the mechanism is not yet well understood (see, e.g., Chirkov, Applied Biochemistry and Microbiology (Translation of Prikladnaya Biokhimiya i Mikrobiologiya) (2002), 38(1), 1-8). Additionally, chitosan is known to impart anti-odor properties (see, for example, WO 1999061079(A1)).

Chitosan is the commonly used name for poly-[1-4]-β-D-glucosamine. Chitosan is chemically derived from chitin which is a poly-[1-4]-β-N-acetyl-D-glucosamine. Including the cell walls of fungi, the exoskeletons of insects and, most commonly from crustaceans. Chitin is inexpensively derived from widely available materials. It is available as an article of commerce from, for example, Primex Corporation (Norway); Biopolymer Engineering, Inc., St. Paul, Minn.; Biopolymer Technologies, Inc., Westborough, Mass.; and CarboMer, Inc., Westborough, Mass.

There are multiple known methods of treating the surfaces of materials with chitosan to prepare antimicrobial articles, such as methods that involve crosslinking or generating reactive groups to attach chitosan to the material surface. In addition, JP01-0342435 discloses an antimicrobial coating agent that includes both chitosan and an emulsion or aqueous dispersion of a synthetic resin selected from among copolymers which include unsaturated carboxylic acids as monomer components and ionomers obtained by partially or totally neutralizing the copolymers with metal ions. The chitosan is mixed at a ratio of about 15 to 70 parts by weight with respect to 100 parts by weight of the aforementioned synthetic resin. The surface is thus a mixture of the chitosan and the synthetic polymer. Its solubility in water of the chitosan used implies that its molecular weight is low, perhaps under 10,000. This reference also teaches away from coating an acidified solution of chitosan onto a film or sheet surface.

Alternatively to chitosan surface treatment, chitosan blends have been prepared that have demonstrated properties of biodegradability or modified mechanical properties. JP05009393 discloses a composite resin particle of chitin and/or chitosan and a soft resin with rubber elasticity that produces a coating film with a soft leathery feel. JP200128228 discloses a biodegradable polymer composition that may include chitosan and a biodegradable polymer reforming agent that is based on a natural rubber. Properties of chitosan/polyethylene-octene elastomer and chitosan/acrylic acid (SS)-grafted-polyethylene-octene elastomer were examined in Wu, J. (Polm. Sci: Pt. A, 41, 3882-3891 (2003)). Mechanical properties such as tensile strength, elongation at break, melting temperature, water resistance, and biodegradability were measured.

U.S. Pat. No. 6,517,933 discloses hybrid polymer materials comprising a set of synthetic building blocks and a set of naturally occurring building blocks with the two sets of building blocks being combined via chemical bonds. Combinations of chitosan and various polymers are proposed where chitosan is reacted with a synthetic polymer. No described materials are produced and no properties of any described materials are measured.

No antimicrobial properties were demonstrated for any of the chitosan blend materials described in the patents and publication above. The methods used to prepare these blends may have resulted in chitosan blends that are not antimicrobial.

There is a need for antimicrobial thermoplastic chitosan blend materials and a need for a process to produce polymer blends with chitosan that imparts antimicrobial properties to the resulting material.

### SUMMARY OF THE INVENTION

The invention discloses chitosan thermoplastic polymer blends and articles composed of said materials. Also provided is a method of producing the antimicrobial chitosan thermoplastic polymer blends.

One aspect is for an antimicrobial thermoplastic polymer blend comprising (a) a water insoluble polymer that contains amino-reactive functional groups, and (b) a chitosan acid salt solution comprising chitosan and at least one aqueous acid.

Amino-reactive functional groups include, for example, metal ions, ammonium ions, anhydrides, carboxylic acids or carbonates, sulfonic acids or sulfonates, isocyanates, epoxides, acid chlorides, and enones, and combinations thereof.

The water insoluble polymer can be, for example, a homopolymer, random copolymer, block copolymer, graft copolymer, or polymer blend.

The antimicrobial thermoplastic polymer blend can further comprise an ionomer. Preferred ionomers include, for example, ionomers of ethylene/acrylic acid copolymer or of ethylene/methacrylic acid copolymer; a perfluorinated sulfonate or carboxylate polymer; a sulfonated polystyrene; a sulfonated ethylene-propylene terpolymer; a sulfonated polyester; or a sulfonated polyamide.

The antimicrobial thermoplastic polymer blend can further comprise one or more metal salts.

Preferred aqueous acids include, for example, acetic acid, valeric acid, formic acid, tartaric acid, citric acid, hydrochloric acid, sulfuric acid, or combinations thereof.

Another aspect is for a process for preparing an antimicrobial thermoplastic polymer blend comprising:

a) mixing a water insoluble thermoplastic polymer that contains amino-reactive functional groups with a chitosan acid salt solution comprising chitosan and at least one aqueous acid to produce a blend; and

b) optionally drying the blend produced in step a).

A further aspect is for a process for preparing an antimicrobial thermoplastic polymer blend comprising the sequential steps of:

a) melting a water insoluble thermoplastic polymer that contains amino-reactive functional groups;

b) mixing the melted polymer of step a) with a chitosan acid salt solution comprising chitosan and at least one aqueous acid to produce a blend; and

c) optionally drying the blend produced in step b).

### DETAILED DESCRIPTION OF THE INVENTION

Entire disclosures of all references cited are incorporated herein by reference.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The invention provides chitosan blend thermoplastic polymers with antimicrobial properties. The polymers are produced by combining a chitosan acid salt with a polymer that contains amino-reactive functional groups. Also provided are materials produced using this method and articles comprising such material.

The following definitions and abbreviations are to be use for the interpretation of the claims and the specification. “Amino-reactive groups” as used herein refers to chemical functionalities that readily undergo chemical reaction with an NH₂ group. Examples include charged species such as metal ions, ammonium ions, anhydrides, carboxylic acids or carbonates, sulfonic acids or sulfonates, isocyanates, epoxides, acid chlorides, and enones.

The term “a blend” refers to a mixture of at least two components. A blend may be a dispersion, a mixture where one component is solubilized in the other, or a combination that is partially solubilized and partially dispersed.

The term “thermoplastic” refers to a polymer that softens when exposed to heat and returns to its original condition when cooled to room temperature.

The term “antibacterial” as used herein means bactericidal as is commonly known in the art. The number of bacteria present after contact with an antibacterial material is substantially reduced from the number initially present. The number of bacteria present is normally measured as colony-forming units.

The term “antimicrobial” as used herein means antibacterial as well as having fungicidal and antiviral activities as is commonly known in the art.

Polymers for Chitosan Blending

Polymers useful in the present invention include, for example, those which are water insoluble and contain amino-reactive functional groups. The water insoluble polymer can be, for example, random copolymer, block copolymer, graft copolymer, or polymer blend. One suitable polymer type includes graft copolymers comprising a graft monomer and a backbone polymer, such as, but not limited to, those described in U.S. Pat. No. 4,026,967, in which the graft monomers include thermally stable unsaturated carboxylic anhydrides and dianhydrides, and the backbone polymers are preferably polymers of ethylene and copolymers derived from ethylene and C₃-C₈ α-olefins, including copolymers of at least one olefin with other monomers. Examples of suitable graft monomers for use in the present invention include methacrylic acid, acrylic acid, glycidyl methacrylate, 2-hydroxy ethylacrylate, 2-hydroxy ethyl methacrylate, diethyl maleate, monoethyl maleate, di-n-butyl maleate, maleic anhydride, maleic acid, fumaric acid, itaconic acid, itaconic anhydride, dodecenyl succinic anhydride, 5-norbornene-2,3-anhydride, and nadic anhydride (3,6-endomethylene-1,2,3,6-tetrahydrophthalic anhydride). Fumaric acid, maleic anhydride, and glycidyl methacrylate are particularly preferred graft monomers. Examples of suitable backbone polymers are polypropylene; polyethylene (e.g., high density polyethylene (HDPE), low density polyethylene (LDPE), linear low density polyethylene (LLDPE), metallocene-catalyzed polyethylene, very low density polyethylene (VLDPE), ultrahigh molecular weight polyethylene (UHMWPE), high performance polyethylene (HPPE)); copolymers of ethylene and propylene; copolymers derived from ethylene or propylene and at least one monomer chosen from propylene, methyl acrylate, ethyl acrylate, n-butyl acrylate, methyl methacrylate, acrylic acid, methacrylic acid and carbon monoxide; and copolymers of olefins with a diolefin, such as a copolymer of ethylene, or of propylene, or of ethylene and other olefins, with: linear aliphatic nonconjugated dienes of at least six carbon atoms (such as 1,4-hexadiene) and other dienes, conjugated or not, such as norbornadiene, dicyclopentadiene, ethylidene norbornene, butadiene, and the like. Other suitable backbone polymers are copolymers of ethylene and tetrafluoroethylene, such as Tefzel® ETFE fluoropolymer resin available from E. I. du Pont de Nemours and Company (Wilmington, Del., USA). One example of a commercially available graft copolymer suitable for use in the present invention is Bynel® 4033, a maleic anhydride grafted HDPE available from E. I. du Pont de Nemours and Company (Wilmington, Del., USA).

Another type of polymer suitable for use in the present invention is a copolymer of an olefin with an acrylic, methacrylic, and/or butylacrylic acid. Ethylene is the preferred olefin. An example of a commercially available material is Nucrel® ethylene acid copolymer resin available from E. I. du Pont de Nemours & Company (Wilmington, Del.).

Other polymers suitable for use in the present invention are ionomers. The term “ionomer” as used herein refers to a polymer with inorganic salt groups attached to the polymer chain (Encyclopedia of Polymer Science and Technology, 2nd ed., H. F. Mark and J. I. Kroschwitz eds., vol. 8, pp. 393-396). Two typical ionomer structures are shown below. 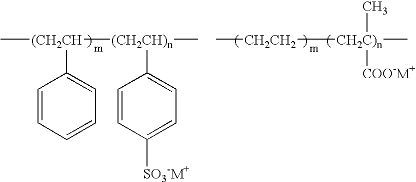
where the ratio of m to n is on the order of 10 to 100; that is, typically only about 1 to 9% of the repeat units contain ionic groups. Ions M are typically metal ions like lithium, sodium, lithium, or zinc but can be other cations (for example, ammonium). Typically, an acid form of the polymer is made first and then neutralized to the desired degree with base containing the desired metal ions. Partially neutralized poly(ethylene-co-methacrylic acid) and partially neutralized poly(ethylene-co-acrylic acid) are examples of ionomers, as is sulfonated polystyrene. Some examples of commercially available ionomers are Surlyn® thermoplastic resin and Nafion® perfluorinated sulfonic acid membranes, available from E. I. du Pont de Nemours and Company (Wilmington, Del., USA); Flemion® perfluorocarboxylate ionomers developed by Asahi Glass Company in Japan; and a sulfonated ethylene-propylene terpolymer from Exxon. Polyesters and polyamides that have been polymerized with a low level of sulfonated comonomer to enhance textile dyeability (see, e.g., U.S. Pat. Nos. 5,559,205; 5,607,765; and 3,389,549) and sulfonated aromatic polyamides (see, e.g., U.S. Pat. Nos. 3,567,632 and 4,595,708) such as those used in reverse osmosis membranes and other selective separation membranes are also suitable polymers for the present invention.
Chitosan

Chitosan is the common name for poly-[1-4]-β-D-glucosamine. Chitosan is chemically derived from chitin which is a poly-[1-4]-β-N-acetyl-D-glucosamine. Chitin is treated with strong alkalis to remove acetyl groups producing chitosan. Depending on the specific treatment of chitin, chitosan may vary in the degree of deacetylation. Chitosan is generally insoluble in water, but dissolves in dilute solutions of organic acids such as acetic, formic, tartaric, valeric, lactic, glycolic, and citric acids and also dissolves in dilute mineral acids such as hydrochloric and sulfuric acids. Preparations of unusually short chitosan polymers (of low molecular weight, that is, less than about 10,000 Daltons) are soluble in water. Typical chitosan preparations have varying molecular weights of individual species.

Aqueous solutions of low molecular weight chitosans in acid salt form and varying molecular weight chitosan acid salt solutions are suitable for the present invention, as are dry chitosan acid salts. Chitosan salts formed by the reaction of chitosan and an acid are suitable for the present invention. The acid may be an organic acid for forming, for example, chitosan acetate, chitosan formate, chitosan acrylate, chitosan butyrate, chitosan valerate, chitosan lactate, chitosan glycolate, and chitosan proprionate. The acid may be an inorganic acid forming, for example, chitosan phosphate, chitosan hydrochloride, and chitosan sulfate. Mixtures of different chitosan salts are also suitable. Chitosan salts may have a wide range of molecular weights due to different chain lengths of the polymer. A chitosan salt preparation having a mixture of molecular weights is suitable for the present invention.

Chitosan for Blending

In the present invention, chitosan is blended as a dry acid salt or an acid salt solution. For example, a solution of chitosan dissolved in dilute acetic acid or another acid as described above may be blended. Other acids may be added with the chitosan in addition to or replacing some or all of the acetic acid. Less volatile acids (such as fatty acids, including for example stearic acid and oleic acid) may be added additionally or partially replace another acid to produce a more stable thermoplastic product. These acids are known to modify the mechanical properties of ionomers and can be chosen for inclusion according to the targeted end use of the chitosan/copolymer blended material. For example, some end uses require specific degrees of hardness, flexibility, heat-sealability, and toughness, among other properties in addition to antimicrobial properties. Fatty acids may be chosen to provide these desired properties, as well as to help disperse the chitosan to achieve optimal antimicrobial properties.

Various molecular weight chitosans may be used in the present invention. Lower molecular weight chitosans may be made into higher concentration solutions so that less solvent needs to be removed during an extrusion process. However, blends with higher molecular weight chitosans may be more durable over time in a wet environment. An alternative is to provide concentrated dispersions of chitosan salts in another solvent, such as an alcohol, for the extrusion process. The alcohol is removed more easily than water, and the final product has the same composition. Higher chitosan concentration solutions are also more viscous, providing improved blending properties, but lower pumping ease. One skilled in the art will know how to determine the viscosity of chitosan solution that is compatible with a particular manufacturing process.

Polymer Together with Chitosan Parameters

Levels of acid in the polymer used in chitosan salt blending may vary, with higher levels of acid providing better blends than polymers with lower acid levels. For example, in ethylene/alkyl acrylate copolymers the relative amount of the alkyl acrylate comonomer incorporated with ethylene can, in principle, vary broadly from a few weight percent up to as high as about 40 weight percent of the total copolymer or even higher. Similarly, the choice of the alkyl group can, again in principle, vary from a simple methyl group up to a six-carbon atom alkyl group with or without significant branching. The relative amount and choice of the alkyl group present in the alkyl acrylate ester comonomer can be viewed as establishing how and to what degree the resulting ethylene copolymer is to be viewed as a polar polymeric constituent in the thermoplastic composition and what level of acid is present.

Polymers with levels of ethylene methyl acrylate (EMA) between about 15% and 24 wt % are preferred. Even though the chitosan may exist as phase-separated domains (so that every amine group is not associated with a polymeric acid group), it is desirable to choose a polymer acid level comparable to or greater than the available amine level. This can be quantified in an acid/amine molar ratio. For example, a chitosan preparation may have 0.0062 moles of amine groups per gram of chitosan. If this chitosan is added at 10 wt % of a polymer, 0.00062 moles/gram will be available. If a 15 wt % EMA copolymer is chosen as the blending polymer, the acid functional groups are present at a level of 0.0017 moles/gram, resulting in an acid/amine ratio of 2.7. If a copolymer with 24% EMA is used, the acid groups are calculated to be present at a level of 0.0028 moles/gram of polymer resulting in an acid/amine ratio of 4.5. A partially neutralized ionomer may be used, as presented in the Examples, which has un-neutralized acid groups calculated to be 0.00049 moles/gram. Using the chitosan preparation having 0.0062 moles of amine groups per gram of chitosan results in an acid/amine ratio of 0.8. The acid/amine ratio of 0.8 provided antimicrobial properties to the resulting blended chitosan/copolymer. Thus a range of acid/amine levels may be used in the present invention.

The level of chitosan present in the final product is based on a number of considerations. The first consideration is to provide sufficient antimicrobial properties for the application. A second consideration is the cost of chitosan. For commercial production it is desirable to have a level of chitosan content that is as low as possible to maintain desirable antimicrobial properties. A typical level of chitosan is about 10%, however, this may vary depending upon additional considerations such as the longevity of antimicrobial activity desired, and the type of end product use, such as involving washing or abrasion. In products requiring longevity of use or harsh conditions, higher levels of chitosan may be called for.

Blending Methods

Blending of a chitosan salt or salt solution and a polymer may be by any method known to one skilled in the art. Methods include, for example, press-blending and extrusion. In press-blending, typically the materials to be blended are combined and pressed together repeatedly under changing configurations, as described in the Examples.

Blending by Extrusion

Parameters for extrusion blending of chitosan salts or salt solutions with polymers are variable depending on the specific materials used and the desired outcome. Several parameters are listed below:

1) The extrusion temperature profile is chosen to be high enough to melt and process the polymer, but as low as feasible to minimize degradation of chitosan. For example, an EMA copolymer with 24 wt % methyl acrylate is generally extruded at 180° C.

2) The rate of addition of the chitosan solution is adjusted in ratio to the polymer feed rate to achieve the desired blend ratio in the final product.

3) The chitosan concentration in solution can be as high as possible to minimize the amount of water to be removed later. The upper limit to the chitosan concentration is determined by the solution viscosity that can be made and pumped. For example, when a chitosan with a molecular weight of 75,000 Daltons is used, the maximum usable concentration is about 8%. Lower molecular weight chitosans may make solutions with higher concentrations. A stoichiometric amount of acid is added with the water to ensure that the chitosan dissolves. For example, for every gram of chitosan, at least about 0.38 grams of acetic acid are added.

One skilled in the art will be adept at ascertaining the specific parameters needed for the above variables, as well as for additional variables, for the desired processing and outcome.

Chitosan/polymer blends prepared by the methods of the present invention exhibit antimicrobial properties and are expected to inhibit odor development as well. Said antimicrobial properties may, optionally, be further enhanced by including metal salts in the blends. Metal salts useful for the present invention include, for example, zinc sulfate, copper sulfate, silver nitrate, or other water-soluble zinc, copper, and silver salts or mixtures of these. The metal salts are typically included by incorporating a dilute (0.1% to 5%) solution of the salt in water in the blending process, including dry metal salt in blending, or by including metal salt in the chitosan solution.

Articles Including Chitosan Blended Polymers

Articles comprising the chitosan blended polymeric material of the present invention may be in the form of or comprise a film, membrane, laminate, fiber, filament, yarn, knit fabric, woven fabric, nonwoven fabric, pellet, coating, or foam. Articles may be prepared by any means known in the art, such as, but not limited to, methods of injection molding, extruding, blow molding, thermoforming, solution casting, film blowing, knitting, weaving, or spinning.

The following are examples of articles wherein it is desirable to reduce microbial growth in or on the article in the end-use for which the particular article is commonly used.

The articles of the invention include packaging for food, personal care (health and hygiene) items, and cosmetics. By “packaging” is meant either an entire package or a component of a package. Examples of packaging components include, but are not limited, to packaging film, liners, absorbent pads packaging, shrink bags, shrink wrap, trays, tray/container assemblies, caps, adhesives, lids, and applicators. Such absorbent pads, shrink bags, shrink wrap, and trays of the present invention are particularly useful for packaging meat, poultry, and fish.

The package may be in any form appropriate for the particular application, such as a can, box, bottle, jar, bag, cosmetics package, or closed-ended tube. The packaging may be fashioned by any means known in the art, such as by extrusion, coextrusion, thermoforming, injection molding, lamination, or blow molding.

Some specific examples of packaging include, but are not limited to, bottles, tips, applicators, and caps for prescription and non-prescription capsules and pills, solutions, creams, lotions, powders, shampoos, conditioners, deodorants, antiperspirants, and suspensions for eye, ear, nose, throat, vaginal, urinary tract, rectal, skin, and hair contact; lip product packaging; and caps.

Examples of applicators include lipstick, chapstick, and gloss; packages and applicators for eye cosmetics, such as mascara, eyeliner, shadow, dusting powder, bath powder, blusher, foundation and creams; and pump dispensers and components thereof. These applicators are used to apply substances onto the various surfaces of the body, and reduction of microbial growth will be beneficial in such applications.

Other forms of packaging components included in the present invention include drink bottle necks, replaceable caps, non-replaceable caps, and dispensing systems; food and beverage delivery systems; baby bottle nipples and caps; and pacifiers. Where a liquid, solution or suspension is intended to be applied, the package may be fashioned for application in a form for dispensing discrete drops or for spraying of droplets. The invention will also find use in pharmaceutical applications fashioned as inhalers.

Examples of end-use applications, other than packaging, in the area of food handling and processing that benefit from antimicrobial functionality and wherein microbial growth is reduced in the particular end-use of the consumer are components of food handling and processing equipment, such as temporary or permanent food preparation surfaces; conveyer belt assemblies and their components; equipment for mixing, grinding, crushing, rolling, pelletizing, and extruding and components thereof; heat exchangers and their components; drains and their components; equipment for transporting water such as, but not limited to, buckets, tanks, pipes, and tubing; and machines for food cutting and slicing and components thereof. Where such equipment components are metal, a coating of a chitosan blended polymeric material could be applied to the metal surface. In one embodiment, the equipment component is a screw for mixing and/or conveying that is an element in a single-screw or twin-screw extruder, such as, but not limited to, an extruder used for food processing; and the polymer coating comprises an ionomer blended with chitosan.

Articles of the present invention can also be used in or as items of apparel, such as a swimsuit, undergarment, shoe component (for example, a woven or nonwoven shoe liner or insert), protective sports pad, child's garment, or medical garment (such as a gown, mask, glove, slipper, bootie, or head covering). Such garments particularly benefit from the inhibition of odor development.

Articles of the present invention can also be used in or as medical materials, devices, or implants, such as bandages, adhesives, gauze strips, gauze pads, medical or surgical drapes, syringe holders, catheters, sutures, IV tubing, IV bags, stents, guide wires, prostheses, orthopedic pins, dental materials, pacemakers, heart valves, artificial hearts, knee and hip joint implants, bone cements, vascular grafts, urinary catheter ostomy ports, orthopedic fixtures, pacemaker leads, defibrillator leads, ear canal shunts, cosmetic implants, ENT (ear, nose, throat) implants, staples, implantable pumps, hernia patches, plates, screws, blood bags, external blood pumps, fluid administration systems, heart-lung machines, dialysis equipment, artificial skin, ventricular assist devices, hearing aids, and dental implants.

In the hygiene area, articles of the present invention include personal hygiene garments such as diapers, incontinence pads, panty liners, sanitary napkins, sports pads, tampons and their applicators; and health care materials such as antimicrobial wipes, baby wipes, personal cleansing wipes, cosmetic wipes, diapers, medicated wipes or pads (for example, medicated wipes or pads that contain an antibiotic, a medication to treat acne, a medication to treat hemorrhoids, an anti-itch medication, an anti-inflammatory medication, or an antiseptic).

Articles of the present invention also include items intended for oral contact, such as a baby bottle nipple, pacifier, orthodontic appliance or elastic bands for same, denture material, cup, drinking glass, toothbrush, or teething toy.

Additional child-oriented articles that benefit through comprising the chitosan blended polymeric material of the present invention include baby bottles, baby books, plastic scissors, toys, diaper pails, and a container to hold cleansing wipes.

Household articles of the present invention include telephones and cellular phones; fiberfill, bedding, bed linens, window treatments, carpet, flooring components, foam padding such as mat and rug backings, upholstery components (including foam padding), nonwoven dryer sheets, laundry softener containing sheets, automotive wipes, household cleaning wipes, counter wipes, shower curtains, shower curtain liners, towels, washcloths, dust cloths, mops, table cloths, walls, and counter surfaces.

The current invention is also useful in reducing or preventing biofilm growth on selective separation membranes (for example, pervaporation, dialysis, reverse osmosis, ultrafiltration, and microfiltration membranes), and air and water filters that can be made from chitosan blended polymeric material.

The current invention is also useful in providing an antifouling surface on boat components such as, but not limited to, boat hulls and components thereof, and boat motors and components thereof. A film of a chitosan blended polymeric material could be heat sealed to the boat component's surface.

Devices used in fluid, (e.g., water), transportation and/or storage can also benefit from the antimicrobial chitosan blended polymeric material of the invention. Exemplary devices include, but are not limited to, pipes and tanks. The pipe or tank itself may be made from the chitosan blended polymeric material, or a chitosan blended polymeric material may be applied to the inner and/or outer surface to provide antimicrobial functionality.

### EXAMPLES

The invention is further defined in the following Examples, in which all parts and percentages are by weight and degrees are Celsius. These Examples, while indicating preferred embodiments of the invention, are given by way of illustration only. From the above discussion and these Examples, one skilled in the art can ascertain the essential characteristics of this invention, and, without departing from the spirit and scope thereof, can make changes and modifications to adapt the invention to various usages and conditions.

The meaning of abbreviations used is as follows: “min” means minute(s), “hr” means hour, “μL” means microliter(s), “ml” means milliliter(s), “nm” means nanometer(s), “mm” means millimeter(s), “cm” means centimeter(s), “μm” means micrometer(s), “mM” means millimolar, “M” means molar, “mils” means one one-thousandth of an inch, “g” means gram(s), “gsm” means grams per square meter, “cfu/ml” means colony forming units per milliliter.

Materials and Methods:

The chitosan used in this study was material commercially available under the registered trademark ChitoClear® from Primex Corporation of Norway. The material was used as purchased.

Surlyn® ionomer was obtained from E. I. du Pont de Nemours and Company (Wilmington, Del., USA) (DuPont).

The degree of N-deacetylation of the chitosan samples was ascertained by proton and carbon 13 NMR spectroscopy to be over 85%. The molecular weight of the samples was equal to or greater than about 75,000.

Chitosan/polymer blends were tested for antimicrobial properties by the Shake Flask Test for Antimicrobial Testing of Materials using the following procedure:

1. Inoculate a single, isolated colony from a bacterial or yeast agar plate culture in 15-25 ml of Trypticase Soy Broth (TSB) in a sterile flask. Incubate at 25-37° C. (use optimal growth temperature for specific microbe) for 16-24 hr with or without shaking (select appropriate aeration of specific strain).

2. Dilute the overnight bacterial or yeast culture into sterile phosphate buffer (see below) at pH 6.0 to 7.0 to obtain approximately 10⁵ colony forming units per ml (cfu/ml). The total volume of phosphate buffer needed will be 50 ml×number of test flasks (including controls). For filamentous fungi, prepare spore suspensions at 10⁵ spores/ml. Spore suspensions are prepared by gently resuspending spores from an agar plate culture that has been flooded with sterile saline or phosphate buffer. To obtain initial inoculum counts, plate final dilutions (prepared in phosphate buffer) of 10⁻⁴ and 10⁻³ onto Trypticase Soy Agar (TSA) plates in duplicate. Incubate plates at 25-37° C. overnight.

3. Transfer 50 ml of inoculated phosphate buffer into each sterile test flask containing 0.5 g of material to be tested. Also, prepare control flasks of inoculated phosphate buffer and uninoculated phosphate buffer with no test materials.

4. Place all flasks on a wrist-action shaker and incubate with vigorous shaking at room temperature. Sample all flasks periodically and plate appropriate dilutions onto TSA plates. Incubate at 25 to 37° C. for 8 or more hrs and count colonies.

5. Report colony counts as the number of Colony Forming Units per ml (cfu/ml).

Stock phosphate buffer:

| | |
|---|---|
| Monobasic Potassium Phosphate: | 22.4 g |
| Dibasic Potassium Phosphate: | 56.0 g |
| Deionized Water: | Bring up volume to 1000 ml |

Adjust the pH of the phosphate buffer to pH 6.0 to 7.0 with either NaOH or HCl, filter, sterilize, and store at 4° C. until use. The working phosphate buffer is prepared by diluting 1 ml of stock phosphate buffer in 800 ml of sterile deionized water.

### Example 1

### Preparation of Chitosan Salt Blended Surlyn® Ionomer

An extruder blend of chitosan and Surlyn® ionomer was simulated by press-blending chitosan acetate salt and Surlyn® ionomer according to the following procedure: (1) a chitosan solution was made in dilute acetic acid; (2) the solution was spread onto a Surlyn® ionomer film and dried, leaving the acetate salt of chitosan; (3) the two-layer film was folded and pressed repeatedly to disperse and/or dissolve the chitosan acetate salt into the Surlyn® ionomer.

Chitosan Solution

The chitosan used in this work was Chitoclear® TM656 chitosan powder that was made from shrimp shells (Primex Corporation, Haugesund, Norway). It had an average molecular weight of 75,000 Dalton and was more than 95% deacetylated. Solutions containing 4% chitosan were made by slurrying 30 g of dry chitosan powder in 405 g of water. Then, under vigorous agitation, additional water (300 g) mixed with acetic acid (15 g) was added. The solution was stirred for 3 more min to yield a smooth syrup-like solution. This solution was stored for 1 week before being used for blending.

Forming a Chitosan Film on the Surlyn® Ionomer

The Surlyn® ionomer used in this work was Surlyn® 1605 (DuPont) made into a film by extrusion onto a chill roll. The film had a basis weight of 47 gsm and was corona-treated to be easily wetted by the chitosan solution.

A chitosan acetate salt coating was formed on a Surlyn®) ionomer film of 30.5 cm by 45.7 cm by placing 15 ml of 4% chitosan solution (described above) at the top of the film and drawing it down using a #30 wire-wound rod. The wet film was allowed to air dry until it was dry to touch (about 1 hr), and a second coat was applied over the first in the same manner. This was also allowed to air dry (about 1 hr), and a third coat of chitosan solution was applied over the first two. This film was then allowed to air dry over night (about 18 hrs) and was placed into a plastic bag to minimize additional loss of acetate ions from equilibration with moisture in the air. The resulting coated film was examined by microscopy and found to be approximately 7 microns thick, indicating that the chitosan layer is about 11% of the total thickness.

Press-Blending

A 4-inch by 4-inch square of the two-layer chitosan-acetate salt/Surlyn®) 1605 film was cut and folded into a 1-inch by 1-inch square with the chitosan-acetate salt side facing in. This square was placed between two sheets of Teflon®-coated aluminum foil (Tri-Foil T303, Saint-Gobain Performance Plastics Corp., Paris, France) and loaded between platens of a heated Carver press (Carver, Inc., Wabash, Ind., USA) The sample was pressed at 100° C., and held at 5000 pounds of press load for 5 secs. The result was a two-inch diameter disk, 10 mils in thickness. This was removed and folded into a 0.75 inch by 0.75 inch square and re-pressed the same way as above. The procedure was repeated until a total of 10 hot pressings were made.

### Example 2

### Preparation of Chitosan Powder Blended Surlyn® Ionomer

A control for Example 1 was made by press-blending chitosan powder into Surlyn® 1605 film. The same Chitoclear® TM656 chitosan powder was used, but this had never been dissolved in dilute acetic acid as was done in Example 1. Four 2-inch by 2-inch squares of Surlyn® 1605 film were cut, which weighed a total of 0.5 grams. Chitosan powder (0.13 g) was sprinkled onto three of these squares, which were stacked and topped with the fourth to make a sandwich structure, containing approximately 20% chitosan. This sandwich structure was then pressed in the heated Carver press as described in Example 1. After each pressing, the disk was folded and repressed, for a total of ten pressings at 100° C.

Another control for Example 1 was made by hot-pressing a film of Surlyn® 1605 without any chitosan. This sample was pressed, folded and repressed for a total of ten pressings at 100° C., as in Examples 1 and 2 above.

### Example 3

### Antimicrobial Evaluation of Prepared Surlyn® Ionomer Blends

The three differently prepared films described in Examples 1 and 2 were assayed in a standard shake-flask test (described in Materials and Methods above) for 8 hrs to detect antimicrobial activity against Salmonella choleraesuis, ATCC 9239, known to be difficult for chitosan to kill. The control Surlyn® film blended with chitosan powder and the control Surlyn® film blended with no additive produced no log reduction at all as compared to the inoculated sample with no test material present. However, the test sample made from chitosan-acetate salt showed a 2.0 log reduction in the number of cfu/ml after 8 hrs.

Thus making a polymer blend using a chitosan salt retains the antimicrobial activity of chitosan in the resulting material, while making the blend with chitosan powder does not provide any antimicrobial activity in the resulting material.

### Example 4

### Preparation of Chitosan Salt Blended Polymer in an Extruder

The following discussion exemplifies how a similar product is made in an extruder.

This blend is made in a vented twin-screw extruder, using the following procedure:
Ethylene acrylate copolymer, or other acid-containing or partially neutralized acid-containing thermoplastic polymer, is fed to the twin-screw extruder. The extrusion temperature profile is chosen to be high enough to melt and process the polymer, but as low as feasible to minimize degradation of chitosan (to be added in the next step). For example, an EMA copolymer with 24 wt % methyl acrylate is extruded at 180° C.
A solution of chitosan dissolved in dilute acetic acid (or another acid) is added along with polymer pellets. The rate of this component addition is adjusted in ratio to the polymer feed rate to achieve the desired blend ratio in the final product. A typical level of chitosan in the final product is 10%. A stoichiometric amount of acid is added with the water to ensure that the chitosan dissolves. For example, for every gram of chitosan, at least 0.38 grams of acetic acid are added.
The water and excess volatile acid is removed through one or more vacuum ports in the extruder during the blending process.
Exiting the extruder, the new blend is either cut into pellets by conventional techniques for further processing, or it is immediately formed into a part, for example by injection molding. Films, fibers and nonwoven fabrics are examples of “parts” that can be made directly from the extrusion process.

1. A polymer blend comprising (a) a water insoluble polymer that contains amino-reactive functional groups, and (b) a chitosan acid salt solution comprising chitosan and at least one aqueous acid wherein the amino-reactive functional group includes one or more metal ions, ammonium ions, anhydrides, carboxylic acids or carbonates, sulfonic acids or sulfonates, isocyanates, epoxides, acid chlorides, enones, or combinations thereof and the polymer is a homopolymer, random copolymer, block copolymer, graft copolymer, or blend of polymers.

2. The polymer blend of claim 1 wherein the polymer is the graft copolymer comprising (a) a graft monomer that is a thermally stable unsaturated carboxylic anhydride or dianhydride, and (b) a backbone polymer including homopolymer of ethylene, homopolymer of propylene, copolymer derived from ethylene and one or more C₃-C₈ α-olefins, or a copolymer derived from propylene and one or more C₄-C₈ α-olefins; and the graft monomer includes methacrylic acid, acrylic acid, glycidyl methacrylate, 2-hydroxy ethylacrylate, 2-hydroxy ethyl methacrylate, diethyl maleate, monoethyl maleate, di-n-butyl maleate, maleic anhydride, maleic acid, fumaric acid, itaconic acid, itaconic anhydride, dodecenyl succinic anhydride, 5-norbornene-2,3-anhydride, or nadic anhydride (3,6-endomethylene-1,2,3,6-tetrahydrophthalic anhydride).

3. The polymer blend of claim 2 wherein the backbone polymer is (a) polypropylene; (b) polyethylene including high density polyethylene, low density polyethylene, linear low density polyethylene, metallocene catalyzed polyethylene, very low density polyethylene, ultrahigh molecular weight polyethylene, or high performance polyethylene; (c) copolymers of ethylene and propylene; (d) copolymer derived from ethylene or propylene and at least one monomer including methyl acrylate, ethyl acrylate, n-butyl acrylate, methyl methacrylate, acrylic acid, methacrylic acid, or carbon monoxide; (e) copolymer of olefin with a diolefin wherein the olefin preferably includes ethylene, propylene, or ethylene with one or more other olefins and the diolefin preferably includes linear aliphatic nonconjugated diene of at least six carbon atoms, norbornadiene, dicyclopentadiene, ethylidene norbornene, or butadiene; or (f) copolymer of ethylene and tetrafluoroethylene.

4. The polymer blend of claim 1 further comprising an ionomer, a metal salt, a fatty acid, or mixtures thereof wherein the ionomer includes ionomer of ethylene/acrylic acid copolymer, ionomer of ethylene/methacrylic acid copolymer, ionomer of perfluorinated sulfonated, ionomer of carboxylate polymer, ionomer of sulfonated polystyrene, ionomer of sulfonated ethylene-propylene terpolymer, ionomer of sulfonated polyester, or ionomer of sulfonated polyamide; and the metal salt includes water-soluble zinc salt, water-soluble copper salt, water-soluble silver salt, or mixtures thereof.

5. The polymer blend of claim 3 further comprising an ionomer, a metal salt, a fatty acid, or mixtures thereof wherein the further comprising an ionomer and a metal salt are each as recited in claim 4.

6. The polymer blend of claim 5 wherein the diolefin includes linear aliphatic nonconjugated dienes of at least six carbon atoms, norbornadiene, dicyclopentadiene, ethylidene norbornene, or butadiene.

7. The polymer blend of claim 4 wherein the polymer is a copolymer of ethylene with acrylic acid, methacrylic acid, or butylacrylic acid.

8. The polymer blend of claim 6 wherein the water insoluble polymer is a copolymer of ethylene with acrylic acid, methacrylic acid, or butylacrylic acid.

9. The polymer blend of claim 7 wherein the aqueous acid comprises one or more acids including acetic acid, valeric acid, formic acid, tartaric acid, citric acid, lactic acid, glycolic acid, hydrochloric acid, or sulfuric acid.

10. A process for preparing a polymer blend comprising (a) mixing a water insoluble thermoplastic polymer that contains amino-reactive functional groups with a chitosan acid salt solution comprising chitosan and at least one aqueous acid and optionally additive to produce a polymer blend; and (b) optionally drying the polymer blend or (c) melting a water insoluble thermoplastic polymer comprising one or more amino-reactive functional groups to produce a melted polymer, mixing the melted polymer with a chitosan acid salt solution comprising chitosan and at least one aqueous acid and optionally additive to produce a polymer blend, and optionally drying the polymer wherein
the additive includes an ionomer, a fatty acid, a metal salt, or mixtures thereof each as characterized in claim 4;
the polymer blend is characterized in claim 1; and
the aqueous acid is one or a combination of acids including acetic acid, valeric acid, formic acid, tartaric acid, citric acid, hydrochloric acid, lactic acid, glycolic acid, or sulfuric acid.

11. The process of claim 10 wherein the polymer blend is as recited in claim 4.

12. The process of claim 10 wherein the polymer blend is as recited in claim 5.

13. The process of claim 10 wherein the polymer blend is as recited in claim 8.

14. The process of claim 10 wherein the polymer blend is as recited in claim 9.

15. An article comprising a polymer blend wherein the article includes film, membrane, laminate, knit fabric, woven fabric, nonwoven fabric, fiber, filament, yarn, pellet, coating, or foam and the polymer blend is as recited in claim 1.

16. The article of claim 15 wherein
the article is a package, packaging component, food or beverage dispensing system, toy, diaper pail, container for cleansing wipes, toothbrush, food handling and processing equipment, item of apparel, household article, bandage, adhesive, medical or surgical drape, medical device or implant, air or water filter, boat component, or fluid transportation or storage device;
the package is a bottle, box, jar, can, bag, close-ended tube, cosmetics package, or inhaler and the package preferably contains a food or a beverage;
the packaging component is in the form of liner, lid, adhesive, replaceable or disposable container cap, film, shrink wrap, shrink bag, tray, tray/container assembly, absorbent pad for packaging, applicator, drink bottle neck, food dispensing system, or beverage dispensing system;
the applicator is a pump dispenser or component thereof, mascara wand, medicated pad or wipe, cosmetics brush, dropper, tip, lipstick applicator, eyeliner applicator, or eye shadow applicator.
the food handling and processing equipment is a conveyor belt assembly and component thereof; temporary and permanent food preparation surface; equipment for mixing, grinding, crushing, rolling, pelletizing, and extruding and component thereof; heat exchanger and its component; drain and its component; bucket, tank, pipe, and tubing; or machine for food cutting and slicing and component thereof.
the equipment for extruding comprises a screw for mixing and/or conveying and wherein the polymer coating comprises an ionomer; and
the boat component is a boat hull, a component of a boat hull, a boat motor, or a component of a boat motor.

17. The article of claim 16 wherein the polymer blend is as recited in claim 8.

18. The article of claim 16 wherein the polymer blend is as recited in claim 9.

19. A package for meat, poultry, or fish comprising the shrink wrap, shrink bag, tray, absorbent pad for packaging, and combinations thereof with an article as recited in claim 17.

20. The package of claim 19 wherein the article is as recited in claim 18.
